(19) United States
(12) Patent Application Publication
(10) Pub. No.: US 2004/0022815 A1
(43) Pub. Date: Feb. 5, 2004

(54) Novel pharmaceutical composition of ceftiofur

(75) Inventors: Kour Chand Jindal, Chennai (IN); Majid Razzak, Auckland (NZ); Nilendu Sen, Chennai (IN)

Correspondence Address: OLIFF & BERRIDGE, PLC, P.O. BOX 19928, ALEXANDRIA, VA, 22320, US

(73) Assignees: ORCHID HEALTH CARE, Tamilnadu (IN); ANCARE NEW ZEALAND LTD., Auckland (NZ)

(21) Appl. No.: 10/211,580

(22) Filed: Aug. 5, 2002

(51) Int. Cl. A61K 31/545; A61K 9/00

(52) U.S. Cl. 424/400; 514/200

(57) ABSTRACT

The present invention relates to a pharmaceutical oily suspension comprising cephalosporin antibiotic or its pharmaceutically acceptable salt, at least a biocompatible oil, a wetting agent, a dispersing agent and a resuspendibility enhancer, said suspension has improved properties, such as resuspendibility and chemical stability and process thereof.

### FIELD OF THE INVENTION

This invention relates to a novel pharmaceutical composition comprising cephalosporin antibiotic or its pharmaceutically acceptable salts. Particularly the invention relates to oily suspension of Ceftiofur or its pharmaceutically acceptable salts. More particularly the present invention provides oily suspension of Ceftiofur hydrochloride, which has improved properties, such as resuspendibility and chemical stability.

### BACKGROUND OF THE INVENTION

Ceftiofur is a third generation Cephalosporin antibiotic, which is administered to cattle and swine for control of bacterial infections of the respiratory tract. It is used in veterinary medicine as both, the sodium salt and the hydrochloride salt. It is administered intramuscularly to cattle and swine. It is also intended to be used as crystalline free acid for intramuscular and subcutaneous administration in cattle and swine. Ceftiofur is poorly absorbed after oral administration while rapidly absorbed after intramuscular administration. The Ceftiofur hydrochloride intramuscular injection is an oily suspension.

A suspension is a particular class or type of dispersion system in which the internal or suspended phase is dispensed uniformly with mechanical agitation through out the external phase, called the suspending medium or vehicle. The internal phase, consisting of a homogenous or heterogeneous distribution of solid particles having a specific range of sizes, these particles are maintained uniformly in time throughout the suspending vehicle with the aid of a single, or a particular combination of suspending agents.

The three general classes of pharmaceutical suspensions are orally administered suspensions, externally applied suspensions (topical) and injectable (parenterals) suspensions (Ref: A Martin & P Bustamante, Coarse Dispersion in physical pharmacy 45^{th} edn. Lea and Febiger, Philadelphia, 1993, PP 117-124). The Parenteral suspensions are designed for intramuscular, intradermal, intralesional, intraarticular or subcutaneous administration. Common vehicles for parenteral suspensions include preserved sodium chloride solution or a parenterally acceptable vegetable oil.(Ref: J P Partnoff, E M Cohen & M M Henlay, Development of Parenteral and Sterile ophthalmic suspensions, Bull. Parenter. Drug Assoc 31: 136-143 (1977)).

The stability of suspensions is complicated by the fact that the physical stability of pharmaceutical suspensions and the factors affecting such stability are equally important to chemical stability. This is based on the fact that since a suspension exists in more than one state (liquid and solid), there are different ways in which the system can undergo either chemical or physical change (Ref: T Higuchi, some physical chemical aspects of suspension formulation, J. Am. Pharm. Assoc. Sci Ed; 47: 657-660 (1958)).

Haines & Martin, Hiestand and Econow & Coworkers (Ref: J. Pharm. Sci., 61; 268-272, (1972) and J. Pharm. Sci., 52; 757-762, 1031-1038, (1963)) and are generally credited with establishing the structured particle concept or flocculated pharmaceutical suspension.

The following definitions will prove useful in differentiating the three closely related terms; flocculation, agglomeration and coagulation.

Flocculation refers to the formation of a loose aggregation of discrete particles held together in a network-like structure either by physical absorption of macromolecules, bridging during chemical interaction, or when the longer range Vander Waals forces of attraction exceeds the shorter range force of repulsion.

In agglomeration, a large number of particles are closely bound together as aggregates either in a dry or liquid state.

Coagulation or flocculation refers to the massing of particles in a liquid state alone and sometimes in the form of a fluid gel structure.

The main advantages of the stable flocculated systems are as follows:

1. The aggregates tend to break up easily under the application of a small amount of shear stress such as gentle agitation of a bottle or vials or by the flow through a small orifice (hypodermic needle and for syringe)

2. In contrast to deflocculated systems, the stable flocculation will settle rapidly and may be easily resuspended even after standing for prolonged time period of storage.

3. The stable flocculation can be produced if required by employing aseptic techniques using vehicle components that are safe for intramuscular injection.

There are several methods of producing flocculated pharmaceutical suspensions. The choice of method depends on the properties of the drug and the class of suspension desired.

The relevant prior art methods, which teach adaptation of diverse methods of preparation, are as follows.

U.S. Pat. No. 5,736,151 claims a pharmaceutical composition comprising Ceftiofur HCl & water. This patent claims a pharmaceutical composition comprising a Ceftiofur HCl, a biocompatible oil (selected from canola oil, corn oil, cottonseed oil, olive oil, peanut oil, sesame oil, soybean oil, safflower oil, coconut oil, sunflower oil and palm oil) and water present in an amount between 0.25% and 20.20% of the composition as a flocculating agent in order to have better resuspendibility than the formulation having no water.

U.S. Pat. No. 4,902,683, discloses crystalline hydrochloride and hydrobromide salts of the cephalosporin antibiotic, Ceftiofur, processes for their manufacture, and pharmaceutical compositions thereof. This patent discloses preparations for systemic administration, which include carriers, vehicles, diluents, surfactants, excipients, preservatives, isotonic agents and the like.

In order to achieve an improved pharmaceutical composition, applicants have conducted lot of experiments with different composition, studied their resuspendability, chemical stability, and finally come out with a new composition with desired results. The present invention is based on Applicants observation that when a resuspendibility enhancer is added to the oily suspension the resuspendibility of the suspended particles is improved dramatically, thus giving a better physical stability to the oily suspension. Applicants have also observed the chemical stability of the oily suspension is improved with the decrease in the moisture content. In addition, applicants have initiated formulation trials to stabilize the suspension physically and chemically using agents such as polyoxyl hydrogenated castor oil, polyoxyl castor oil, glycerol, propylene glycol, polyethylene glycol, alcohols and the like. Different formulations were kept in stoppered cylinders and the rate of sedimentation was calculated. The comparative sedimentation ratio (Ratio of sedimentation volume to total volume of the suspension) of different batches of Ceftiofur hydrochloride is shown in FIG. 1.

### OBJECTIVE OF THE INVENTION

The main objective of the present invention is to develop a suspension of Ceftiofur or its pharmaceutically acceptable salts which has high physico-chemical stability and easy to administer.

Another objective of the present invention is to provide a physically stable suspension using resuspendibility enhancers and a chemically stable suspension by reducing the moisture content.

Still another objective of the present invention is to provide a suspension with adequate dispersion of the particles in the vehicle.

Yet another objective of the present invention is to provide a suspension which does not form caking of the dispersed particles in the sediment which is difficult to redisperse.

### SUMMARY OF THE INVENTION

Accordingly, the present invention relates to an oily suspension comprising ceftiofur or its pharmaceutically acceptable salts, at least a biocompatible oil, a wetting agent, a dispersing agent, a resuspendibility enhancer.

Preferably the said composition comprises of 0.025% to 1.0% by weight of wetting agent, 0.05% to 2.5% by weight of dispersing agent and 0.05% to 10% by weight of a resuspendibility enhancer.

More preferably, the said composition comprises of 0.025% to 0.1% by weight of a wetting agent, 0.05% to 0.5% by weight of dispersing agent and 0.05% to 10% by weight of a resuspendibility enhancer.

In a further related aspect, the invention provides a process for the preparation of novel oily suspension of ceftiofur or its pharmaceutically acceptable salt, said process comprising steps of:

i) heating a biocompatible oil to a temperature in the range of 40° C. to 160° C., based on the dosage form,

ii) adding a wetting agent, to step (i) liquid and allowing to cool with stirring,

iii) adding a dispersing agent and a resuspendibility enhancer to step (ii) mixture with stirring, if required,

iv) stirring the mixture at about 160° C. up to 2 hrs,

v) adding Ceftiofur or its pharmaceutically acceptable salt to step (iv) mixture and mixing well with stirring,

vi) homogenizing step (v) mixture using a suitable homogenizer and

vii) filling in the primary packaging components.

### BRIEF DESCRIPTION OF ACCOMPANYING DRAWING

FIG. 1 represents a graphical representation of sedimentation rate of various formulae using different flocculating agents.

### DETAILED DESCRIPTION OF THE INVENTION

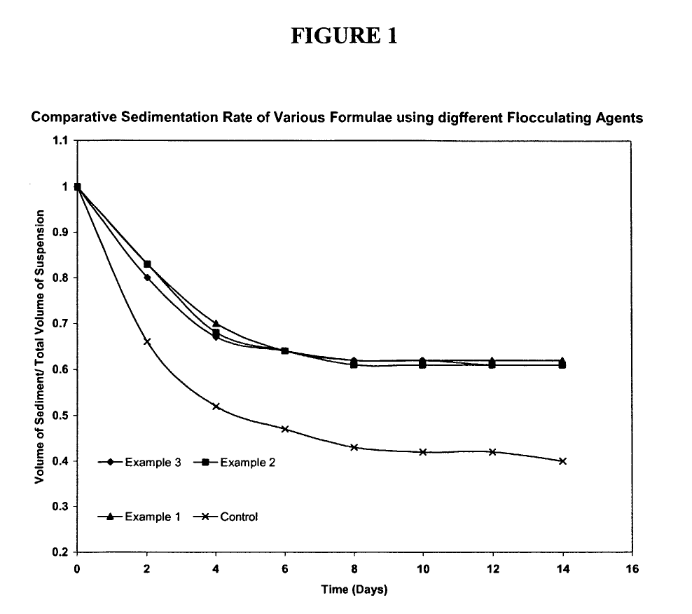

In accordance, applicants have initiated formulation trials to stabilize the suspension physically and chemically using agents such as polyoxyl hydrogenated castor oil, polyoxyl castor oil, glycerol, propylene glycol, polyethylene glycol, alcohols and the like. Different formulations were kept in stoppered cylinders and the rate of sedimentation was calculated. The comparative sedimentation ratio (Ratio of sedimentation volume to total volume of the suspension) of different batches of Ceftiofur hydrochloride is shown in FIG. 1.

In an embodiment of the present invention provides an oily suspension comprising ceftiofur or its pharmaceutically acceptable salts, at least a biocompatible oil, a wetting agent, a dispersing agent, a resuspendibility enhancer and optionally acceptable excipients.

In another embodiment of the present invention, there is provided an injectable suspension, which has improved physical stability using resuspendibility enhancers.

In still another embodiment of the present invention there is provided an injectable suspension, which has greater chemical stability by reducing the moisture content.

In an embodiment of the present invention, the pharmaceutically acceptable salts of Ceftiofur are selected from sodium, hydrochloride or hydrobromide, preferably Ceftiofur hydrochloride.

In an embodiment of the present invention Ceftiofur or its pharmaceutically acceptable salt may be present in amount of 1% to 20% by weight of the suspension, preferably in amount of 1% to 10%. Further, the Ceftiofur hydrochloride may be present in an amount of 10 mg to about 200 mg/ml, preferably in an amount of 10 mg to about 100 mg/ml.

In an embodiment of the present invention, the biocompatible oil used may be selected from the group consisting of monoglyceride, diglyceride, triglyceride medium chain succinic acid triglyceride, corn oil, cottonseed oil, olive oil, sesame oil, soybean oil, safflower oil, coconut oil, sunflower oil or palm oil, preferably cottonseed oil, peanut oil, corn oil is used.

In yet another embodiment of the present invention, the wetting and dispersing agents used in the suspension are selected from lecithin, fatty acid ester of sorbitan or glycerol.

In yet another embodiment of the present invention, the resuspendibility enhancer is selected from polyoxyl hydrogenated vegetable oil, polyoxyl vegetable oil, glycerol, propylene glycol, polyethylene glycols.

In yet another embodiment of the present invention, the oily suspension of Ceftiofur hydrochloride is used for oral, topical or parenteral administration.

In yet another embodiment of the present invention, the oily suspension can be sterilized by sterilization techniques known in the art.

The oily suspension of the present invention has improved resuspendibility. The addition of the resuspendibility enhancers improves the particle interaction, which result in a “loose” particle aggregation so when the suspension is shaken the particles can separate to some extent and a uniform dose can be obtained.

The present invention is detailed by the examples below, which are provided by way of illustration only and should not be considered to limit the scope of the invention.

### EXAMPLE 1

Composition:

Ceftiofur (as Ceftiofur HCl): 50 g

Lecithin: 0.5 g

Sorbitan monooleate: 0.5 g

Propylene glycol: 5 g

Cottonseed oil q.s. to 1000 ml

800 m of Cottonseed oil is heated to above 100° C. and lecithin is added and stirred well until dissolved. The oil containing the wetting agent is then cooled with continuous stirring. Sorbitan monooleate is added next with agitation. Next the required amount of Propylene glycol is added and mixed well with stirring. Sterile Ceftiofar Hydrochloride is added and mixed for 15 to 45 minutes. Volume is made up to 1000 ml by adding sufficient amount of cottonseed oil. The suspension is then homogenized using a suitable homogenizer. It is then filled in vials, closed with a stopper, sealed and sterilized.

### EXAMPLE 2

Composition:

Ceftiofur (as Ceftiofur HCl): 50 g

Lecithin: 0.5 g

Sorbitan monooleate: 0.5 g

Polyethylene Glycol-400: 5 g

Cottonseed oil q.s. to 1000 ml

800 ml of Cottonseed oil is heated to above 100° C. and lecithin is added and stirred well until dissolved. The oil containing the wetting agent is then cooled with continuous stirring. Sorbitan monooleate is added next with agitation. Next the required amount of Polyethylene glycol 400 is added and mixed well with stirring. Sterile Ceftiofur Hydrochloride is added and mixed for 15 to 45 minutes. Volume is made up to 1000 ml by adding sufficient amount of cottonseed oil. The suspension is then homogenized using a suitable homogenizer. It is then filled in vials, closed with a stopper, sealed and sterilized.

### EXAMPLE 3

Composition:

Ceftiofur (as Ceftiofur HCl): 50 g

Lecithin: 0.5 g

Sorbitan monooleate: 0.5 g

Polyoxyl 40 hydrogenated castor oil: 1 g

Cottonseed oil q.s. to 1000 ml

800 ml of Cottonseed oil is heated to above 100° C. and lecithin is added and stirred well until dissolved. The oil containing the wetting agent is then cooled with continuous stirring. Sorbitan monooleate is added next with agitation. Next the required amount of Polyoxyl 40 hydrogenated castor oil is added and mixed well with stirring. Sterile Ceftiofur Hydrochloride is added and mixed for 15 to 45 minutes. The suspension is then homogenized using suitable homogenizer. Volume is made up to 1000 ml by adding sufficient amount of cottonseed oil. It is then filled in vials, closed with a stopper, sealed and sterilized.

### EXAMPLE 4

Composition:

Ceftiofur (as Ceftiofur HCl): 50 g

Lecithin: 0.5 g

Sorbitan monooleate: 0.5 g

Polyoxyl 40 hydrogenated castor oil: 1 g

Cottonseed oil q.s. to 1000 ml

800 ml of Cottonseed oil is heated to above 100° C. and lecithin is added and stirred well until dissolved. Sorbitan monooleate is added next with agitation. Next the required amount of Polyoxyl 40 hydrogenated castor oil is added and mixed well with stirring. The oily mixture is heated to 160° C., for 2 hours and is then cooled. Sterile Ceftiofur Hydrochloride is added and mixed for 15 to 45 minutes. The suspension is then homogenized using suitable homogenizer. Volume is made up to 1000 ml by adding sufficient amount of cottonseed oil. It is then filled in vials, closed with a stopper and sealed.

Chemical Stability and Shelf-Life

The stability of suspensions, and ultimately their shelf life, is based on both chemical and physical stability. The chemical stability is assessed to insure that the product does not become subpotent during shelf life. The chemical stability of the current formulation has been studied extensively at different temperatures and different time periods. The results for example 3 are shown below:

| | Initial Assay | | | | |
|---|---|---|---|---|---|
| | (% of | | 1 | 2 | 3 |
| Assay | Label claim) | 2 wks | Month | Months | Months |
| 25° C./60%RH | 99.16 | — | 98.68 | — | 98.61 |
| Assay (% of | | | | | |
| Label claim) | | | | | |
| 40° C./75%RH | 99.16 | — | 97.11 | 97.32 | 96.75 |
| Assay (% of | | | | | |
| Label claim) | | | | | |
| 60° C. | 99.16 | 99.22 | — | — | — |

Resuspendibility or Physical Stability

Resuspendibility is a very important parameter that determines the dosage accuracy in the dispensed volume of the suspension during the entire shelf life of the product. If the resuspension is not adequate the initial doses shall be subpotent as some small amount of drug remains at the bottom of the container.

The invention describes of such formulation where the resuspendibility is achieved effortlessly to get uniform dispersion.

Freeze-Thaw Study

Samples were subjected to four cycles of 0° C. & 40° C. each of 48 hours and then observed for any physical changes in the suspension characteristics and also analyzed for chemical stability. The results are shown below:

| Test | Example No. 1 | Example No. 3 |
|---|---|---|
| Physical Appearance | No significant change | No significant change |
| Appearance after | No significant change | No significant change |
| Freeze-thaw cycling | | |
| Initial Assay | 99.79 | 99.16 |
| (% Label Claim) | | |
| Assay after Freeze-thaw | 97.81 | 95.66 |
| cycling (% Label Claim) | | |

1. A novel oily suspension of Ceftiofur or its pharmaceutically acceptable salt, said composition comprising at least a biocompatible oil, a wetting agent, a dispersing agent and a resuspendibility enhancer.

2. The oily suspension as claimed in claim 1, wherein Ceftiofur or its pharmaceutically acceptable salt is present in an amount of 1% to 20% by weight of the suspension.

3. The oily suspension as claimed in claim 1, wherein Ceftiofur or its pharmaceutically acceptable salt is present preferably in amount of 1% to 10%.

4. The oily suspension as claimed in claim 1, wherein said composition comprises of 0.025% to 1.0% by weight of wetting agent, 0.05% to 2.5% by weight of dispersing agent and 0.05% to 10% by weight of resuspendibility enhancer.

5. The oily suspension as claimed in claim 4, wherein said composition preferably comprises of 0.025% to 0.1% w/v of wetting agent, 0.05% to 0.5% w/v of dispersing agent and 0.05% to 10% w/v of resuspendibility enhancers.

6. The oily suspension as claimed in claim 1, wherein the pharmaceutically acceptable salt of Ceftiofur is selected from sodium, hydrochloride or hydrobromide.

7. The oily suspension as claimed in claim 1, wherein the biocompatible oil used is selected from the group consisting of monoglyceride, diglyceride, triglyceride medium chain succinic acid triglyceride, corn oil, cottonseed oil, olive oil, sesame oil, soybean oil, safflower oil, coconut oil, sunflower oil or palm oil.

8. The oily suspension as claimed in claim 1, wherein the wetting and dispersing agent used is selected from lecithin, fatty acid ester of sorbitan or glycerol.

9. The oily suspension as claimed in claim 1, wherein the resuspendibility enhancer used is selected from polyoxyl hydrogenated vegetable oil, polyoxyl vegetable oil, glycerol, propylene glycol or polyethylene glycol.

10. The oily suspension as claimed in claim 1, which has acceptable or good physical stability by using resuspendibility enhancers.

11. The oily suspension as claimed in claim 1, which has better chemical stability due to reduced moisture content.

12. The oily suspension as claimed in claim 1, wherein the suspension is administered orally, topically or parenterally.

13. A process for the preparation of novel oily suspension, which comprises:
i) heating a biocompatible oil to a temperature in the range of 40° C. to 160° C., based on the dosage form,
ii) adding a wetting agent to step (i) liquid and allowing to cool, with stirring,
iii) adding a dispersing agent and a resuspendibility enhancer to step (ii) mixture with stirring, if required
iv) stirring the mixture of step (iii) at about 160° C. up to 2 hrs
v) adding Ceftiofur or its pharmaceutically acceptable salt to step (iv) mixture and mixing well with stirring,
vi) homogenizing step (v) mixture using a suitable homogenizer, and
vii) filling in the primary packaging components.
